(19) United States
(12) Patent Application Publication
(10) Pub. No.: US 2010/0010069 A1
(43) Pub. Date: Jan. 14, 2010

(54) Extended Length Borane Phosphonate Nucleic Acid Compounds

(75) Inventors: Heather Brummel McCuen, Boulder, CO (US); Agnieszka B. Sierzchala, Boulder, CO (US); Marvin H. Caruthers, Boulder, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of Colorodo, Denver, CO (US)

(21) Appl. No.: 12/282,319

(22) PCT Filed: Mar. 9, 2007
(86) PCT No.: PCT/US07/06132

§ 371 Date: Sep. 18, 2009

Related U.S. Application Data
(60) Provisional application No. 60/780,975, filed on Mar. 10, 2006.

(51) Int. Cl. A61K 31/7052; C07H 19/16; C07H 19/06; C07H 1/00; A61P 43/00

(52) U.S. Cl. 514/44 R; 536/26.5; 536/26.7; 536/26.8; 536/25.3

(57) ABSTRACT

The present invention provides a novel method for solid-phase phosphoramidite based synthesis of borane phosphonate DNA. Also provided are novel phosphoramidite molecules, novel extended length borane phosphonate nucleic acid compounds, and methods of use thereof.

### BACKGROUND OF THE INVENTION

For some time now, oligodeoxyribonucleotides (ODNs) bearing internucleotide borane phosphonate linkages have been of considerable interest for applications in diagnostic and therapeutic areas because they mimic natural DNA in various biological processes. The problem with this analog is the lack of high yielding, chemical methods for its synthesis.

To date, the most successful synthesis approach has been conversion of deoxyoligonucleotide H-phosphonates, via silylation followed by boranation, to oligomers having exclusively borane phosphonate internucleotide linkages. Results using unprotected bases suggest that 10 mers can be prepared but with low yields and variable purity. However neither mixed sequences having all four bases in high purity nor ODNs having both borane phosphonate and phosphate linkages are possible via this chemistry. Recently, an alternative method featuring mononucleotide borane phosphonates and a phosphotriester strategy has been used to prepare, for the first time, borane phosphonate dinucleotides having all four bases. Unfortunately the coupling yields range from 72% to 92%, which is insufficient for the synthesis even of 10 mers.

The present invention provides solutions to these and other problems in the art of borane phosphonate nucleic acid chemistry.

### SUMMARY OF THE INVENTION

In one aspect, the present invention provides extended length borane phosphonate nucleic acid compounds having any or all four nucleotide bases (i.e. A, G, C, T) at any desired position, and any number of desired borane phosphonate internucleotide linkages at any desired position.

In another aspect, the present invention provides novel N-trityl phosphoramidite molecules.

In another aspect, the present invention provides a general method of synthesizing a borane phosphonate DNA. The method includes contacting the 5′ hydroxyl of a solid phase 2′deoxy nucleic acid with an N-trityl phosphoramidite molecule thereby forming an N-trityl solid phase 2′ deoxy nucleic acid. The resulting N-trityl solid phase 2′ deoxy nucleic acid is contacted with a boronation reagent thereby forming an N-trityl phosphonium borane solid phase 2′ deoxy nucleic acid. The N-trityl phosphonium borane solid phase 2′ deoxy nucleic acid is then contacted with a fluoride ion thereby removing the silyl protecting group and forming a 5′-OH N-trityl phosphonium borane solid phase 2′ deoxy nucleic acid. The 5′-OH N-trityl phosphonium borane solid phase 2′ deoxy nucleic acid may optionally be extended using one or more phosphoramidite coupling, 5′ deprotection, and oxidation cycles. The 5′-OH N-trityl phosphonium borane solid phase 2′ deoxy nucleic acid is then contacted with an acidic reagent thereby removing the trityl protecting group and forming a 5′-OH phosphonium borane solid phase 2′ deoxy nucleic acid. The 5′-OH phosphonium borane solid phase 2′ deoxy nucleic acid is contacted with a phosphorous center deprotecting reagent to form a 5′-OH borane phosphonate solid phase 2′ deoxy nucleic. Finally, the 5′-OH borane phosphonate solid phase 2′ deoxy nucleic is contacted with a basic reagent thereby forming a borane phosphonate DNA.

In another aspect, the present invention provides a method of hybridizing the borane phosphonate nucleic acid compound of the present invention to a complementary nucleic acid. The method includes contacting the complementary nucleic acid sequence with the borane phosphonate nucleic acid. The complementary nucleic acid includes a nucleic acid sequence having at least 50% base complementation relative to the borane phosphonate nucleic acid sequence.

In another aspect, the present invention provides pharmaceutical compositions. The pharmaceutical composition includes a pharmaceutically acceptable excipient and a borane phosphonate nucleic acid compound of the present invention.

### BRIEF DESCRIPTION OF THE DRAWINGS

FIG. 1. 5′-O-Silyl-2′-deoxynucleoside-3′-phosphoramidites and borane phosphonate ODNs. Silyl=benzhydroxy-bis-(trimethoxysilyloxy)silyl. B′=protected Base. B=cytosine, thymine, adenine, and guanine. X,Y=combinations of phosphate and borane phosphonate linkages.

FIG. 2. Reverse phase HPLC analysis of the reaction mixture from the synthesis of compound 10. Inset A: Gel electrophoresis results from total reaction mixtures. Lanes 1-5, d(TpTp)₄TpT, 7, 8, 9 and 10, respectively (see Table 2 for a definition of compounds 7, 8, 9 and 10. Inset B: Phosphorus NMR of compound 10.

### DETAILED DESCRIPTION OF THE INVENTION

### Abbreviations and Definitions

The abbreviations used herein have their conventional meaning within the chemical and biological arts.

Where moieties are specified by their conventional chemical formulae, written from left to right, they equally encompass the chemically identical moieties that would result from writing the structure from right to left, e.g., —CH₂O— is equivalent to —OCH₂—.

The term “alkyl,” by itself or as part of another substituent, means, unless otherwise stated, a straight (i.e. unbranched) or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, including those groups having 10 or fewer carbon atoms. A “lower alkyl” or “lower alkylene” is a shorter chain alkyl or alkylene group, generally having eight or fewer carbon atoms.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

The tern “heteroalkyl,” by itself or in combination with another term, means, unless otherwise stated, a stable straight or branched chain, or cyclic hydrocarbon radical, or combinations thereof, consisting of the stated number of carbon atoms and a heteroatom selected from the group consisting of O, N, P, Si and S, and wherein the nitrogen and sulfur atoms may optionally be oxidized and the nitrogen heteroatom may optionally be quaternized. The heteroatom(s) O, N, P and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂,—S(O)—CH₃, —CH₂—CH₂—S(O)₂— CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′ represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituent moieties for each of the above noted aryl and heteroaryl ring systems may be selected from the group of acceptable substituent moieties described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo” as used herein means an oxygen that is double bonded to a carbon atom.

Each of the above terms (e.g., “alkyl,” “heteroalkyl,” “aryl” and “heteroaryl”) are meant to include both substituted and unsubstituted forms of the indicated radical. Preferred substituent moieties for each type of radical are provided below.

Substituent moieties for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituent moieties, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH_{2OCH}₃, and the like).

Similar to the substituent moieties described for the alkyl radical, substituent moieties for the aryl and heteroaryl groups are varied and may be selected from, for example: halogen, —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituent moieties on adjacent atoms of the aryl or heteroaryl ring may optionally form a ring of the formula -Q′-C(O)—(CRR′)_{q}-Q″-, wherein Q′ and Q″ are independently —NR—, —O—, —CRR′— or a single bond, and q is an integer of from 0 to 3. Alternatively, two of the substituent moieties on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)S—X′—(C″R′″)_{d}—, where s and d are independently integers of from 0 to 3, and X′ is —O—, —NR′—, —S—, —S(O)—, —S(O)₂—, or —S(O)₂NR′—. The substituent moieties R, R′, R″ and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

The term “pharmaceutically acceptable salts” is meant to include salts of the active compounds which are prepared with relatively nontoxic acids or bases, depending on the particular substituent moieties found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, tautomers, geometric isomers and individual isomers are encompassed within the scope of the present invention. The compounds of the present invention do not include those which are known in the art to be too unstable to synthesize and/or isolate.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

In some embodiments, each substituted aryl and/or heterocycloalkyl is substituted with a substituent group, a size limited substituent group, or a lower substituent group. A “substituent group,” as used herein, means a group selected from the following moieties:
(A) —OH, —NH₂, —SH, —CN, —CF₃, oxy, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxy, —OH, —NH₂, —SH, —CN, —CF₃, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxy, —OH, —NH₂, —SH, —CN, —CF₃, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from oxy, —OH, —NH₂, —SH, —CN, —CF₃, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The term “treating” refers to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the methods of the invention successfully treat a patient's delirium by decreasing the incidence of disturbances in consciousness or cognition.

As used herein, a “solid phase” such as a “solid support” is any form of bead, resin or the like, typically used in the art of solid phase synthesis to provide a “handle” whereby a reactant can be made available for synthetic manipulation without the risk of loss yield typically experienced when such syntheses are conducted in solution; the terms “solid support” and “resin” are used interchangeably. The term “solid support” or, “support,” refer to a solid particulate, material to which a nucleic acid, nucleic acid analog, nucleoside or nucleoside analog can be synthesized. Supports used in solid phase synthesis are typically substantially inert and nonreactive with the solid phase synthesis reagents. Methods of using solid supports in solid phase synthesis are well known in the art and may include, but are not limited to, those described in U.S. Pat. Nos. 4,415,732, 4,458,066; 4,500,707, 4,668,777; 4,973,679, and 5,132,418 issued to Caruthers, and U.S. Pat. No. 4,725,677 and Re. 34,069 issued to Koster, each of which are herein incorporated by reference in their entirety for all purposes.

As used herein, “nucleic acid” means single stranded DNA, RNA and derivative thereof Modifications include, but are not limited to, those which provide other chemical groups that incorporate additional charge, polarizability, hydrogen bonding, electrostatic interaction, and functionality to the nucleic acid ligand bases or to the nucleic acid ligand as a whole. Such modifications include, but are not limited to, phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping moieties. A 2′deoxy nucleic acid linker is a divalent nucleic acid compound of any appropriate length and/or internucleotide linkage wherein the nucleotides are 2′deoxy nucleotides.

### Description of the Embodiments

### I. Borane Phosphonate Nucleic Acid Compounds

The present invention provides, for the first time, extended length borane phosphonate nucleic acid compounds having any or all four nucleotide bases (i.e. A, G, C, T) at any desired position, and any number of desired borane phosphonate internucleotide linkages at any desired position.

Extended length borane phosphonate nucleic acid compounds are typically at least 20 nucleotides in length. In some embodiments, the compounds include from 20 to 100 nucleotides. In other embodiments, borane phosphonate nucleic acid compounds are from 20 to 80 nucleotides in length. The borane phosphonate nucleic acid compounds may also be from 20 to 60 nucleotides in length, 20 to 50 nucleotides in length, 20 to 40 nucleotides in length, or 20 to 30 nucleotides in length. In some embodiments, the extended length borane phosphonate nucleic acid compounds are at least 10 nucleotides in length and include at least one borane phosphonate internucleotide linkage.

As discussed above, the borane phosphonate nucleic acid compounds include any number of desired borane phosphonate internucleotide linkages at any desired position. In some embodiments, at least 40% or 45% of the internucleotide linkages of the borane phosphonate nucleic acid compounds are borane phosphonate internucleotide linkages. In other embodiments, at least 50% of the intemucleotide linkages of the borane phosphonate nucleic acid compounds are borane phosphonate internucleotide linkages. The percentage of borane phosphonate internucleotide linkages may also be 55%, 60%, 70%, 80%, 90%, 95%, or 100% of the total internucleotide linkages of the borane phosphonate nucleic acid compound.

Where the percentage of borane phosphonate internucleotide linkages are less than 100%, the remainder of the internucleotide linkages may be any appropriate alternative intemucleotide linkage known to those skilled in the art of nucleic acid chemistry. Appropriate alternative intemucleotide linkages include, but are not limited to, phosphate, thiophosphate, dithiophospate, and methyl phosphonate intemucleotide linkages. In some embodiments, the alternative internucleotide linkage(s) is/are phosphate internucleotide linkages.

In some embodiments, the borane phosphonate nucleic acid compound is a 2′deoxy borane phosphonate nucleic acid compound. In some embodiments, the borane phosphonate nucleic acid compound is attached to a solid support.

### II. Methods of Synthesizing Borane Phosphonate Nucleic Acid Compounds

The compounds of the invention are synthesized by an appropriate combination of generally well known synthetic methods. A more detailed description of certain chemical synthesis techniques are present below in the “Examples” section.

In another aspect, the present invention provides a general method of synthesizing a borane phosphonate nucleic acid compound. The method includes the step of contacting the 5′ hydroxyl of a solid phase nucleic acid (e.g. a solid phase 2′deoxy nucleic acid) with an N-trityl phosphoramidite molecule thereby forming an N-trityl solid phase nucleic acid (e.g. a 2′deoxy nucleic acid) that includes a phosphite triester internucleotide linkage. The N-trityl phosphoramidite molecule typically has the formula:

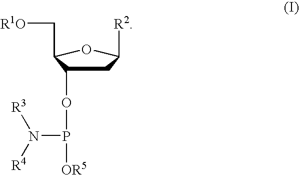

In Formula (I), R¹ is a silyl protecting group. R² is selected from an N3 protected or unprotected thymine, an N2 protected or unprotected guanine, an N6 trityl protected adenine, or an N4 trityl protected cytosine. In some embodiments, R² is an N6 trityl protected adenine or an N4 trityl protected cytosine. In some embodiments, R² is an N2 trityl protected guanine (e.g. trimethoxytrityl). R³ and R⁴ are independently selected from unsubstituted C₁-C₁₀ alkyl, substituted or unsubstituted aryl, or substituted or unsubstituted heterocycloalkyl See S. L. Beaucage and R. P. Iyer, Tetrahedron 48, 2223-2311, 1992.

R⁵ is a fluoride ion compatible phosphorous center protecting group. A “fluoride ion compatible phosphorous center protecting group,” as used herein, refers to a protecting group that is not removed, or is removed minimally, by a fluoride ion used at sufficient concentrations to remove the R¹ silyl protecting group. In some embodiments, R⁵ is an unsubstituted alkyl (e.g. methyl), unsubstituted arylalkyl (e.g. benzyl), or cyanoalkyl (e.g. cyanoethyl).

The resulting N-trityl solid phase 2′ deoxy nucleic acid is contacted with a boronation reagent thereby converting the phosphite triester internucleotide linkage to a PIV phosphonium borane adduct and thus forming an N-trityl phosphonium borane solid phase 2′ deoxy nucleic acid. Any appropriate boronation reagent may be employed. Boronation reagents are selected to efficiently convert phosphite triester internucleotide linkages to PIV phosphonium borane adducts while minimizing or avoiding degradation of other portions of the nucleic acid compound, such as reduction of thymidine base. Useful boronation reagents include, for example, borane-tetrahydrofuran complex, borane-pyridine complex, and borane-diisopropylamine complex. In some embodiments, the boronation reagent may be aided by the use of a boronation activation reagent, such as bis-(trimethylsilyl)-trifluoroacetamide (BSTFA). The N-trityl phosphonium borane solid phase 2′ deoxy nucleic acid is then contacted with a fluoride ion thereby removing the silyl protecting group and forming a 5′-OH N-trityl phosphonium borane solid phase 2′ deoxy nucleic acid.

The 5′-OH N-trityl phosphonium borane solid phase nucleic acid (e.g. 5′-OH N-trityl phosphonium borane solid phase 2′ deoxy nucleic acid) may optionally be extended using one or more phosphoramidite coupling, 5′ deprotection, and oxidation cycles, (as described herein and otherwise known in the art of nucleic acid synthesis). For example, oxidation to a phosphate linkage may be accomplished using a suitable oxidation reagent, such as peroxyanion solution, to produce a mixed internucleotide linkage borane phosphonate nucleic acid compound. Thus, boronation or oxidation to phosphate are compatible and leads to oligomers having any combination of these two linkages.

The 5′-OH N-trityl phosphonium borane solid phase 2′ deoxy nucleic acid or extended nucleic acid typically has the formula:

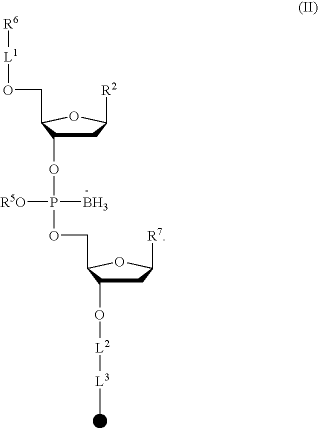

In Formula (II), R⁶ is hydrogen, or a silyl protecting group. L¹ and L² are independently a bond or a 2′deoxy nucleic acid linker. One skilled in the art of nucleic acid chemistry will immediately understand that where the 5′-OH N-trityl phosphonium borane solid phase 2′ deoxy nucleic acid is not extended, R⁶ is hydrogen and L¹ is a bond. Likewise, where the 5′-OH N-trityl phosphonium borane solid phase 2′ deoxy nucleic acid is extended, R⁶ may be hydrogen or a silyl protecting group, depending upon whether a 5′ deprotection step has been performed. Moreover, where the 5′-OH N-trityl phosphonium borane solid phase 2′ deoxy nucleic acid is extended, one skilled in the art will immediately recognize that L¹ is a 2′deoxy nucleic acid linker.

R² and R⁷ are independently selected from an N3 protected or unprotected thymine, an N2 protected or unprotected guanine, an N6 trityl protected adenine, or an N4 trityl protected cytosine. In some embodiments, at least one of R² and R⁷ are an N6 trityl protected adenine or an N4 trityl protected cytosine. As discussed above, R⁵ is a fluoride ion compatible phosphorous center protecting group.

L³ is a base-labile solid support linker. A “base-labile solid support linker” covalently bonds the nucleic acid compound to a solid support and is capable of reacting with a base to release the nucleic acid compound from the solid support. The cleavage of the nucleic acid compound from the solid support by contacting the base-labile solid support linker with a base typically results in a solution phase nucleic acid having a 3′OH moiety. A wide variety of base labile solid support are known in the art and are discussed in detail elsewhere, such as those discussed in Eckstein et al., Oligonucleotides and Analogues: A Practical Approach, (1991).

The solid circle represents a solid support, as defined above.

The 5′-OH N-trityl phosphonium borane solid phase nucleic acid (e.g. 5′-OH N-trityl phosphonium borane solid phase 2′ deoxy nucleic acid) is then contacted with an acidic reagent thereby removing the trityl protecting group and forming a 5′-OH phosphonium borane solid phase nucleic acid (e.g. 5′-OH phosphonium borane solid phase 2′ deoxy nucleic acid). The 5′-OH phosphonium borane solid phase nucleic acid is contacted with a phosphorous center deprotecting reagent to form a 5′-OH borane phosphonate solid phase nucleic acid (e.g. a 5′-OH borane phosphonate solid phase 2′ deoxy nucleic). A “phosphorous center deprotecting reagent,” as used herein, refers to a compound capable of removing the fluoride ion compatible phosphorous center protecting group to form an unprotected phosphonium borane internucleotide linkage. Phosphorus center deprotecting groups are selected to efficiently remove the fluoride ion compatible phosphorous center protecting group while avoiding or minimizing degradation of the remainder of the compound, such as the internucleotide linkages. In some embodiments, the phosphorous center deprotecting reagent is a mild acid, such as acetic acid.

Finally, the 5′-OH borane phosphonate solid phase nucleic acid is contacted with a basic reagent thereby forming a borane phosphonate nucleic acid (e.g. borane phosphonate 2′deoxy nucleic acid). One skilled in the art will immediately recognize that the selection of the basic reagent will depend upon the specific identity of the base-labile solid support linker. In some embodiments, the basic reagent is ammonium hydroxide.

The term “protected,” as used in reference to the protection of a specific nitrogen within a base (e.g. N3 protected thymine, N2 protected guanine, N6 protected adenine), means that a protecting group is attached to the specific nitrogen to prevent the specific nitrogen from reacting with molecules, reactants, reactive functional groups, and the like, during synthesis of the borane phosphonate nucleic acids of the present invention. For example, the N3 protected thymine may be protected with any appropriate protecting group, such as a sterically hindered alkylcarboxy group (e.g. isopropyl, or isobutryl), or carboxyaryl, such as carboxynaphthyl, anisoyl, or benzoyl. Thus, in some embodiments, the N3 protected thymine is an N3 carboxyaryl protected thymine. In other embodiments, the N3 protected thymine is an N3 anisoyl protected thymine or an N3 benzoyl protected thymine. In some embodiments, the N2 protected guanine is an N2 carbamate protected guanine (e.g. 9-fluorenylmethoxycarbonyl) or an N2 trityl protected guanine (e.g. trimethoxytrityl). A carbamate, as used herein, is a nitrogen protecting group wherein the protected nitrogen is attached to an ester linkage (i.e. —NH—C(O)—O—). A trityl is a substituted or unsubstituted triphenylmethyl.

The N6 trityl protected adenine may be an N6 dimethoxytrityl protected adenine, N6 methoxytrityl protected adenine, or N6 trimethoxytrityl protected adenine. Likewise, said N4 trityl protected cytosine is an N4 trimethoxytrityl protected cytosine, N4 dimethoxytrityl protected cytosine, or N4 methoxytrityl protected cytosine. One skilled in the art will immediately recognize that selection of a trityl group will depend upon the synthesis conditions. There are many possible trityl groups available (E. F. Fisher and M. H. Caruthers, Nucleic Acids Res. 11, 1589-1599, 1983; S. L. Beaucage and M. H. Caruthers, U.S. Pat. No. 4,973,679).

In some embodiments, the silyl protecting group is benzhydroxy-bis(trimethylsiloxy)silyl, or bis(trimethylsiloxy)cyclododecyloxysilyl, or tris-(trimethylsiloxy). A silyl protecting group, as used herein, refers to a fluoride ion-labile silyl ether used for S′-OH protection during oligonucleotide synthesis. One skilled in the art will recognize that many silyl protecting groups are useful in the present invention. (See for example Design and Development of New Protecting Groups for RNA Synthesis, Thesis (Ph.D.), Steven A. Scaringe, U. of Colorado, 1996), which is herein incorporated by reference for all purposes.

The 2′ deoxy nucleic acid linker may be a polynucleotide, an oligonucleotide, or a single nucleotide. Typically, the linker is from 1 to 99 nucleotides in length. In some embodiments, the linker is from 1 to 80 nucleotides in length, from 1 to 70 nucleotides in length, from 1 to 60 nucleotides in length, from 1 to 50 nucleotides in length, from 1 to 40 nucleotides in length, from 1 to 30 nucleotides in length, from 1 to 20 nucleotides in length, or from 1 to 10 nucleotides in length. In some embodiments, the 2′ deoxy nucleic acid linker includes a plurality of internucleotide linkages independently selected from a P(IV) posphonium borane adduct internucleotide linkage and a phosphate triester internucleotide linkage.

### III. Methods of Hybridizing the Borane Phosphonate Nucleic Acid Compounds to a Complimentary Nucleic Acid

In another aspect, the present invention provides a method of hybridizing the borane phosphonate nucleic acid compound of the present invention to a complimentary nucleic acid. The method includes contacting the complementary nucleic acid sequence with the borane phosphonate nucleic acid. The complementary nucleic acid includes a nucleic acid sequence having at least 50% base complementation relative to the borane phosphonate nucleic acid sequence. In some embodiments, the complementary nucleic acid comprises a nucleic acid sequence having at least 80%, 90%, 95%, 99%. or 100% base complementation relative to the borane phosphonate nucleic acid sequence.

The hybridization may occur in vivo or in vitro. Thus, in some embodiments, the contacting occurs in a subject, such as an animal (e.g. a mammal such as a human). Where the hybridization occurs in a subject, said method further comprising, before the contacting and/or hybridization, administering the borane phosphonate nucleic acid sequence to the subject. The purpose of administering the borane phosphonate nucleic acid sequence to the subject is typically to treat a disease state in a subject in need of such treatment. In some embodiments, the treatment may be facilitated by antisense action. Thus, in some embodiments, the borane phosphonate nucleic acid compound of the present invention is an antisense nucleic acid.

### IV. Pharameceutical Compositions

In another aspect, the present invention provides pharmaceutical compositions. The pharmaceutical composition includes a pharmaceutically acceptable excipient and a borane phosphonate nucleic acid compound of the present invention.

The pharmaceutical compositions described herein are typically used to treat a disorder or condition using known methods of nucleic acid pharmaceutical therapies, such as antisense methodologies.

In an exemplary embodiment, the pharmaceutical composition includes from 1 to 2000 milligrams a borane phosphonate nucleic acid compound of the present invention. In some embodiments, the pharmaceutical composition includes from 1 to 1500 milligrams of the compound of the borane phosphonate nucleic acid compound of the present invention. In other embodiments, the pharmaceutical composition includes from 1 to 1000 milligrams on a borane phosphonate nucleic acid compound of the present invention.

The borane phosphonate nucleic acid compounds of the present invention can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The compounds of the present invention can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. The borane phosphonate nucleic acid compounds of the present invention can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Thus, the pharmaceutical compositions described herein may be adapted for oral administration. In some embodiments, the pharmaceutical composition is in the form of a tablet. Moreover, the present invention provides pharmaceutical compositions including a pharmaceutically acceptable carrier or excipient and either a borane phosphonate nucleic acid compounds of the present invention, or a pharmaceutically acceptable salt of a borane phosphonate nucleic acid compounds of the present invention.

For preparing pharmaceutical compositions from the borane phosphonate nucleic acid compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain borane phosphonate nucleic acid compounds mixed with a filler or binders such as lactose or starches, lubricants such as talc or magnesium stearate, and, optionally, stabilizers. In soft capsules, the borane phosphonate nucleic acid compounds may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending a borane phosphonate nucleic acid compound in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The borane phosphonate nucleic acid compounds can be delivered by transdermally, by a topical route, formulated as applicator sticks, solutions, suspensions, emulsions, gels, creams, ointments, pastes, jellies, paints, powders, and aerosols.

The borane phosphonate nucleic acid compounds can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The borane phosphonate nucleic acid compounds can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use

In another embodiment, the borane phosphonate nucleic acid compounds are usetul for parenteral administration, such as intravenous (IV) administration or administration into a body cavity or lumen of an organ. The formulations for administration will commonly comprise a solution of the borane phosphonate nucleic acid compound dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of borane phosphonate nucleic acid compound in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the borane phosphonate nucleic acid compound can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the borane phosphonate nucleic acid compound into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

### V. Examples

The following examples are offered to illustrate, but not to limit the claimed invention. The preparation of embodiments of the present invention is described in the following examples. Those of ordinary skill in the art will understand that the chemical reactions and synthesis methods provided may be modified to prepare many of the other compounds of the present invention. Where compounds of the present invention have not been exemplified, those of ordinary skill in the art will recognize that these compounds may be prepared by modifying synthesis methods presented herein, and by using synthesis methods known in the art.

### General Methodology

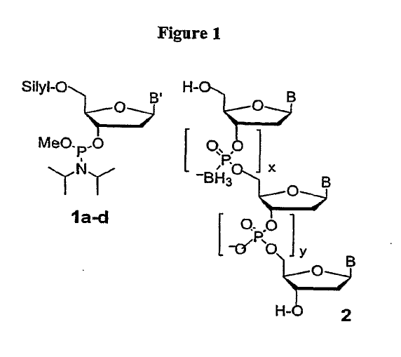

Using appropriately protected 2′-deoxynucleoside phosphoramidites (FIG. 1), a new, high yielding synthesis cycle has been developed. Starting with a 2′-deoxynucleoside attached to polystyrene, the first step is condensation with 1a-d (FIG. 1) in anhydrous acetonitrile and tetrazole to generate a family of dimers having a phosphite triester internucleotide linkage. These dimers are then reacted with either THF•BH₃ or a peroxyanion solution. Removal of 5′-silyl protection with fluoride ion (triethylammonium hydrogen fluoride) generates a family of dinucleotides having any of the four bases and either a PIV phosphonium borane adduct or a phosphate triester linkage. These dimers can then be extended using the same repetitive cycle to generate an ODN of the appropriate length.

Protecting groups are removed sequentially. Initially and with the ODN attached to the support, 80% acetic acid is used to eliminate trityl groups from adenine and cytosine. Next the oligomer is treated with 2 carbamoyl-2-cyanoethylene-1,1-dithiolate to remove methyl protection on internucleotide linkages. Finally concentrated ammonium hydroxide eliminates carbamate and anisoyl groups from guanine and thymine, respectively, cleaves the oligomer from the support, and generates compound 2 (FIG. 1).

### Synthesis of N6-Dimethoxytrityl-2′-deoxyadenosine

2′-Deoxynucleoside (10 mmol) was coevaporated three times with pyridine and dried in vacuo for 12 h. Anhydrous pyridine (50 mL) and chlorotrimethylsilane (50 mmol) were added. After the mixture had been stirred at room temperature for 2 h, dimethoxytrityl chloride (3.7 g, 11 mmol) was added. The reaction was stirred overnight (˜16 h) at room temperature. Water (60 mL) and aqueous ammonium hydroxide (2 mL, 28-30%) were added and the reaction mixture was stirred for 30 min. The crude product was extracted into dichloromethane The organic layer was washed two times with a 5% aqueous solution of sodium bicarbonate and dried with anhydrous sodium sulfate. The organic layer containing product was filtered from salts and purified by column chromatography using chloroform/pyridine (99.9:0.1) and a gradient of methanol (0-6%). Yield 99%. 1H NMR (DMSO-d6) δ 8.42 (s, 1H), 8.32 (s, 1H), 7.28-7.26 (m, 5H), 7.19 (d, 4H), 6.84 (d, 4H), 6.32 (t, 1H), 5.31 (d, 1H), 5.12 (t, 1H), 3.86-3.84 (m, 1H), 3.71 (s, 6H), 3.61-3.47 (m, 2H), 2,79-2.74 (m, 1H), 2.27-2.22 (m, 1H); 13C NMR (DMSO-d6) δ 157.69, 153.68, 151.16, 148.07, 145.34, 140.37, 137.27, 129.77, 128.39, 127.71, 126.47, 121.05, 113.00, 88.04, 84.12, 70.92, 69.61, 61.83, 54.99; HRMS (FAB) calcd for C31H31N5O5 (M+) 553.2325 found 553.2309.

### Synthesis of N4-Trimethoxytrityl-2′-deoxycytidine

2′-Deoxynucleoside (10 mmol) was coevaporated three times with pyridine and dried in vacuo for 12 h. Anhydrous pyridine (50 mL) and chlorotrimethylsilane (50 mmol) were added. After the mixture was stirred at room temperature for 2 h, trimethoxytrityl chloride (3.85 g, 10.5 mmol) was added. The reaction was stirred overnight (˜16 h) at room temperature. Water (60 mL) and aqueous ammonium hydroxide (2 mL, 28-30%) were added, and the reaction mixture was stirred for 30 min. The crude product was extracted into dichloromethane, the organic layer was washed two times with 5% aqueous solution of [symbol] sodium [symbol] . The product was filtered and purified by column chromatography using chloroform/pyridine (99.9:0.1) with a gradient of methanol (0-6%). Yield 95%. 1H NMR (DMSO-d6) δ 8.29 (bs, 1H), 7.69 (d, 1H), 7.10 (d, 4H), 6.82 (d, 4H), 6.21 (d, 1H), 6.04 (t, 1H), 5.17 (d, 1H), 4.94 (t, 1H), 4.16-4.14 (m, 1H), 3.72 (s, 9H), 3.51-3.48 (m, 2H), 2.05-1.86 (m, 2H); 13C NMR(DMSO-d6) δ 163.29, 157.37, 154.07, 139.53, 137.26, 129.78, 112.70, 96.38, 87.18, 84.67, 70.65, 68.92, 61.51, 54.98; HRMS (FAB) calcd for C31H33N3O7 (M+) 559.2319, found 559.2331.

### Synthesis of N2-(9-fluorenylmethoxycarbonyl)-2′-deoxyguanosine

2′-Deoxyguanosine (3.11 g, 11.0 mmol) was twice co-evaporated with 50 mL pyridine and then suspended in 80 mL pyridine. The reaction was started by the dropwise addition of chlorotrimethylsilane (6.5 mL, 75 mmol) with a syringe. The reaction proceeded for 1 h during which the deoxynucleoside was taken into solution. At this point 9-fluorenylmethyl chloroformate (3.5 g, 14.3 mmol) was added and the solution stirred for another 1.5 h. When complete, the reaction mixture was quenched with 20 mL water and stirring for 1 h. Following an aqueous work-up, the crystalline product was dissolved into dichloromethane, filtered and washed with chloroform. The product is a white solid. Yield 65%. 1H NMR (DMSO-d6): δ 2.6 (m, 2H), 3.5 (m, 2H), 3.8 (d, J=2.5 Hz), 4.3 (m, 1H), 4.4 (d, J=6.2 Hz, 2H), 5.3 (m, 1H), 6.2 (t, 1H), 7.3 (t, 2H), 7.4 (t, 2H), 7.8 (d, J=7.0 Hz, 2H), 7.9 (d, J=7.6 Hz, 2H). MS: calcd=489, Found (ESI+)=512 (M+Na+).

### Synthesis of 5′-O-[benzhydroxy-bis(trimethylsilyloxy]silyl-2′-deoxynucleoside

N-protected 2′-deoxynucleoside (10 mmol) was dried in vacuo for 6 h and then dissolved in anhydrous N,N-dimethylformamide (100 mL). Imidazole (20 mmol) was added to the flask and the solution was placed on ice and stirred. Benzhydroxy-bis(trimethylsilyloxy)silyl chloride (10 mmol) was added slowly over 1 h via syringe. The flask was then removed from ice and allowed to stir at room temperature for ˜4 h. The reaction was monitored by TLC and additional aliquots of the silyl chloride (1 mmol) were added until there was no presence of the starting material. Distilled water (60 mL) was added and the solvent was removed in vacuo to a final volume of 50 mL. The remaining solution was dissolved in dichloromethane and rinsed with water saturated sodium chloride and 5% sodium bicarbonate. The organic layer was dried over anhydrous sodium sulfate. The product was filtered and purified by column chromatography. Elution initially was with chloroform/benzene (9:1) followed by a gradient of methanol in chloroform (for N-trityl [symbol] The product eluted in 5-10% methanol.

5′-O-[benzhydroxy-bis(trimethylsilyloxy)silyl]-N3anisoyl-2′-deoxythymidine: yield 65.8%; ¹H NMR (CDCl₃) δ 7.89 (d, 2H), 7.60 (s, 1H), 7.37-7.22 (m, 10H), 6.94 (d, 2H), 6.31 (t, 1H), 5.94 (s, 1H), 4.32-4.29 (m, 1H), 3.90-3.87 (m, 1H), 3.86 (s, 3H), 3.82-3,76 (m, 2H), 2.26-2.20 (m, 1H), 1.98-1.92 (m, 1H), 1.88 (s, 3H), 0.10 (s, 18H); ¹³C NMR (CDCl₃) δ 168.06, 165.30, 163.08, 149.53, 143.97, 143.90, 135.64, 133.26, 128.53, 127.64, 127.60, 126.51, 126.41, 124.44, 114.66, 111.03, 86.78, 85.11, 77.44, 72.18, 63.26, 55.83, 36.71, 13.05, 1.73; HRMS (ESI) calcd for C₃₇H₄₇N₂O₁₀Si₃ (M⁺−H) 763.2544, found 763.2533.

5′-O-[benzhydroxy-bis(trimethylsilyloxy)silyl]-N4trimethoxytrityl-2′-deoxycytidine: yield 43.5%; ¹H NMR (DMSO-d₆) δ 8.40 (bs, 1H), 7.48 (d, 1H), 7.39-7.22 (m, 10H), 7.15 (d, 6H), 6.83 (d, 6H), 6.27 (d, 1H), 6.06 (t, 1H), 5.96 (s, 1H), 5.26 (d, 1H), 4.11-4.05 (m, 1H), 3.80-3.78 (m, 1H), 3.71 (s, 9H), 3.68-3.61 (m, 2H), 2.05-1.98 (m, 1H), 1.74-1.68 (m, 1H). 0.05 (s, 18H); ¹³C NMR (DMSO-d₆) δ 163.28, 157.39, 153.95, 144.06, 138.67, 137.25, 129.81, 129.56, 128.27, 127.16, 125.80, 112.69, 96.48, 86.15, 84.73, 76.07, 70.84, 68.97, 63.28, 54.95, 40.05, 1.43; HRMS (ESI) calcd for C₅₀H₆₂N₃O₁₀Si₃ (M⁺+H) 948.3737, found 948.3725.

5′-O-[benzhydroxy-bis(trimethylsilyloxy)silyl]-N6dimethoxytrityl-2′-deoxyadenosine: yield 67.9%; ¹H NMR (DMSO-d₆) δ 8.30 (s, 1H), 7.89 (s, 1H), 7.34-7.15 (m, 19H), 6.83 (d, 4H), 6.31 (t, 1H), 5.89 (s, 1H), 5.37 (d, 1H), 4.39-4.34 (m, 1H), 3.86-3.83 (m, 1H), 3.80-3.77 (m, 1H), 3.70 (s, 6H), 3.62-3.58 (m, 1H), 2.79-2.74 (m, 1H), 2.28-2.23 (m, 1H), −0.03 and −0.04 (2xs, 18H); ¹³C NMR(DMSO-d₆) δ 157.67, 153.54, 151.14, 148.13, 145.29, 144.00, 140.02, 137.21, 129.66, 128.30, 128.09, 127.61, 126.99, 126.41, 125.75, 120.97, 112.94, 86.58, 83.72, 75.94, 70.61, 69.55, 63.21, 54.93, 38.39, 1.27; HRMS (ESI) calcd for C₅₀H₆₀N₅O₈Si₃ (M⁺+H) 942.3744, found 942.3773.

5′-O-[benzhydroxy-bis(trimethylsilyloxy)silyl]-N2-(9-fluorenylmethoxycarbonyl)-2′-deoxyguanosine: yield 24.3%; ¹H NMR (DMSO-d₆) δ 11.68 (s, 1H), 11.33 (s, 1H), 7.95 (s, 1H), 7.92 (d, 2H), 7.82 (d, 2H), 7.44 (t, 2H), 7.37-7.27 (m, 1OH), 7.22-7.19 (m, 2H), 6.22 (t, 1H), 5.92 (s, 1H), 5.37 (d, 1H), 4.52-4.46 (m, 2H), 4.36-4.31 (m, 2H), 3.86-3.83 (m, 1H), 3.77-3.64 (m, 2H), 2.48-2.25 (m, 2H), 0.00 and −0.01 (2xs, 18H); ¹³C NMR (DMSO-d₆) δ 155.02, 154.44, 148.60, 147.30, 144.02, 143.22, 140.73, 136.92, 128.14, 127.79, 127.06, [symbol] 63.28, 46.08, 39.62, 1.30; HRMS (ESI) calcd for C₄₄H₅₂N₅O₉Si₃ (M⁺+H) 878.3067, found 878.3051.

### Synthesis of 5′-O-silyl-N protected-2′-deoxynucleoside-3′-O-phosphoramidites

Protected 2′-deoxynucleoside (2 mmol) was dried in vacuo for 6 h and then dissolved in anhydrous dichloromethane (20 mL). Methyl tetraisopropylphosphorodiamidite (2.1 mmol) was added and the mixture was stirred. Tetrazole (2 mmol) was added slowly over 1 h and the solution was allowed to stir for an additional 3 h. A small amount of triethylamine (approximately 0.4 mL) was added to neutralize the solution and the solvent was removed in vacuo. The crude product was isolated by chromatography with benzene followed by a gradient of ethyl acetate (0-40 or 100%) in benzene containing 0.1% triethylamine. Triethylamine was excluded during purification of compound 1d in order to prevent the elimination of the Fmoc protecting group.

Compound 1a (B¹=Thy^{an}): yield 67.7%; 31P NMR (CDCl3) δ 150.51, 149.50. ¹³C NMR (CDCl₃) δ168.10, 165.26, 163.07, 149.60, 144.03, 143.93, 135.62, 133.24, 128.52, 128.50, 127.65, 127.57, 126.56, 126.41, 126.39, 124.63, 114.65, 111.06, 86.90, 86.54, 86.48, 85.32, 77.28, 74.07, 63.39, 55.82, 50.65, 50.49, 43.28, 43.22, 43.16, 43.10, 40.21, 24.83, 24.76, 13.04, 1.76, 1.72; HRMS (ESI) calcd for C₄₄H₆₅N₃O₁₁Si₃P (M⁺+H) 926.3659, found 926.3632.

Compound 1b (B¹=C^{TMT}): yield 61.3%; 31P NMR (CDCl3) δ 150.3, 150.06. ¹³C NMR (CDCl₃) δ 165.40, 158.70, 155.46, 144.00, 140.88, 136.83, 129.89, 128.36, 128.33, 127.40, 127.33, 126.43, 126.38, 126.36, 113.64, 94.79, 86.16, 86.11, 76.94, 73.73, 73.56, 73.47, 73.30, 69.69, 62.96, 62.89, 55.28, 50.67, 50.51, 43.15, 43.08, 43.02, 42.96, 40.85, 23.08, 23.06, 1.61; HRMS (ESI) calcd for C₅₇H₇₈N₄O₁₁Si₃P (M⁺+H) 1109.4707, found 1109.4667.

Compound 1c (B¹=A^{DMT}): yield 86.4%; 31P NMR (CDCl3) δ 149.77, δ 149.64. ¹³C NMR (CDCl₃) δ 1158.42, 154.30, 152.41, 148.80, 145.72, 144.26, 144.21, 138.53, 137.76, 130.32, 129.02, 128.41, 128.39, 128.04, 127.34, 126.95, 126.57, 126.51, 126.49, 121.48, 113.30, 86.89, 86.67, 84.63, 84.58, 76.98, 74.37, 74.20, 74.06, 73.89, 70.76, 63.20, 55.41, 50.77, 50.71, 50.60, 50.54, 43.17, 43.04, 39.63, 24.93, 24.86, 24.80, 24.77, 1.72, 1.69; HRMS (ESI) calcd for C₅₇H₇₆N₆O₉Si₃P (M⁺+H) 1103.4713, found 1103.4715.

Compound 1 [symbol] ( [symbol] ): yield 37.1% 31P NMR (CDCl3) δ 149.84, 149.69. ¹³C NMR (CDCl₃) δ 155.86, 153.55, 153.49, 148.47, 148.42, 146.44, 146.38, 144.13, 144.06, 143.02, 142.99, 142.96, 141.53, 137.16, 128.44, 128.42, 128.27, 127.47, 127.39, 126.52, 126.39, 124.98, 121.25, 120.36, 87.07, 86.77, 84.10, 77.07, 74.51, 74.33, 74.06, 68.46, 63.31, 63.24, 50.70, 50.53, 46.81, 43.15, 43.02, 40.19, 24.89, 24.83, 24.76, 1.71, 1.67; 1HRMS (ESI) calcd for C₅₁H₆₈N₆O₁₀Si₃P (M⁺+H) 1039.4036, found 1039.4038.

### Synthesis of N2-trimethoxytrityl-2′-deoxyguanosine

2′-Deoxyguanosine (3.2 g; 11 mmol) was twice co-evaporated with 50 mL pyridine and dried in vacuo for 12 h. Anhydrous pyridine (60 mL) and chlorotrimethylsilane (7.1 mL; 56 mmol) were added. After the mixture was stirred at room temperature for 2 h, trimethoxytrityl chloride (4.3 g; 12 mmol) was added. The reaction was stirred overnight (˜16 h) at room temperature. Pyridine hydrochloride was filtered. Water (60 mL) and aqueous ammonium hydroxide (2 mL) were added and the reaction mixture was stirred for 30 min. The crude product was extracted into dichloromethane, the organic layer was washed two times with 5% aqueous solution of sodium bicarbonate and dried with anhydrous sodium sulfate. The product was filtered and purified by column chromatography using chloroform/pyridine (99.9:0.1) with a gradient of methanol (0-25%). Yield 68.5%.

### Synthesis of 5′-O-[benzhydroxy-bis(trimethylsilyloxy)silyl]-2′-deoxyguanosine

N-Trimethoxytrityl-2′-deoxyguanosine (4.6 g; 7.7 mmol) was dried in vacuo for 12 h and then dissolved in anhydrous N,N-dimethylformamide (80 mL). Imidazole (1.05 g; 15.4 mmol) was added to the mixture and the flask was placed on ice and stirred. Benzhydroxy-bis(trimethylsilyloxy)silyl chloride (4 mL; 9.9 mmol) was added slowly over 2 h via syringe. The flask was then removed from ice and allowed to stir at room temperature for 6 h. The reaction was monitored by TLC. Distilled water (60 mL) was added and solvents were removed in vacuo to a final volume of 50 mL. The remaining solution was dissolved in dichloromethane and rinsed with aqueous solution of 5% sodium bicarbonate saturated with sodium chloride and dried with anhydrous sodium sulfate. The product was filtered and purified by column chromatography using chloroform/pyridine (99.9:0.1) with a gradient of methanol (0-20%). Yield 47.4%.

### Synthesis of 5′-O-silyl-N-trimethoxytrityl-2′-deoxyguanosine 3′-O-phosphoamidite

[symbol] 2′-deoxyguanosine (3.6 g; 3.6 mmol) was dried in vacuo for 12 h and then dissolved in anhydrous dichloromethane (30 mL). Methyl tetraisopropylphosphorodiamidite (1.2 mL; 4 mmol) was added with stirring. 0.4 M solution of tetrazole in acetonitrile (3.2 mL; 3.6 mmol of tetrazole) was added slowly over 2 h and the reaction mixture was stirred for an additional 2 h. A small amount of triethylamine (approximately 0.4 mL) was added to neutralize the solution and the solvents were removed in vacuo. The crude product was isolated by column chromatography with benzene/triethylamine (99:1) followed by gradient of ethyl acetate (0-100%). Yield 75%.

### Synthesis of Oligodeoxynucleotides on a Solid Support

The solid support (5′-O-dimethoxytrityl-2′-deoxythymidine-3′-polystyrene) from Glen Research (LV-PS, 200 nmol) was treated with 3% trichloroacetic acid in dichloromethane prior to automated synthesis. The following synthesis cycle was then used to generate oligodeoxynucleotides having the appropriate sequence and either borane phosphonate or phosphate triester internucleotide linkages.

TABLE 1

| Coupling | 0.1 M Compound 1a, 1b, 1c, or | 5 to column, |
|---|---|---|
| | 1d in acetonitrile and 0.45 M | 60 wait |
| | tetrazole in acetonitrile (1:1) | |
| Wash | Acetonitrile | 30 |
| Boranation | 25 mM BH3•THF in THF | 30 |
| or | | |
| Oxidation | Peroxyanion Solution^{a} | 120 |
| Wash | Tetrahydrofuran | 30 |
| | Dichloromethane | 30 |
| | Acetonitrile | 45 |
| | Dimethylformamide | 35 |
| 5′-Deprotection | 1.1 M HF/1.1 M Triethylamine/ | 25 to column, |
| | 0.2 M N-methyldiethanolamine | 45 wait |
| | in dimethylformamide (pH 9)^{b} | |
| Wash | Dimethylformamide | 40 |
| | Acetonitrile | 60 |
| | 0.4 M Tetrazole in Acetonitrile | 3 |

^{a}Peroxyanion Solution: Solution A, 3% (w/v) aqueous LiOH (10 mL), 1.5 M 2-amino-2-methyl-1-propanol in water (15 mL), and dioxane (17.5 mL). Solution B, m-chloroperbenzoic acid (1.78 g), dioxane (32.5 mL), and aqueous 30% hydrogen peroxide (10 mL). Equal volumes were mixed just prior to synthesis.
^{b}Buffer pH was measured by diluting an aliquot of this solution with water (1:9, v/v) and measuring the pH of the resulting solution.

[symbol]

trimethoxytrityl-2′-deoxyguanosine 3′-O-phosphoramidite for compound 1d, the fully borane phosphonate modified 12 mer, d(AbAbCbGbAbTbAbTbCbGbTbT), was synthesized. Following removal of protecting groups as outlined in paragraph [0089], the total reaction mixture was fractionated by reverse phase HPLC (FIG. 3) with the product being the major peak. The resulting borane phosphonate oligodeoxynucleotide was characterized by phosphorus and boron NMR and by Mass Spectral analysis.

### Results

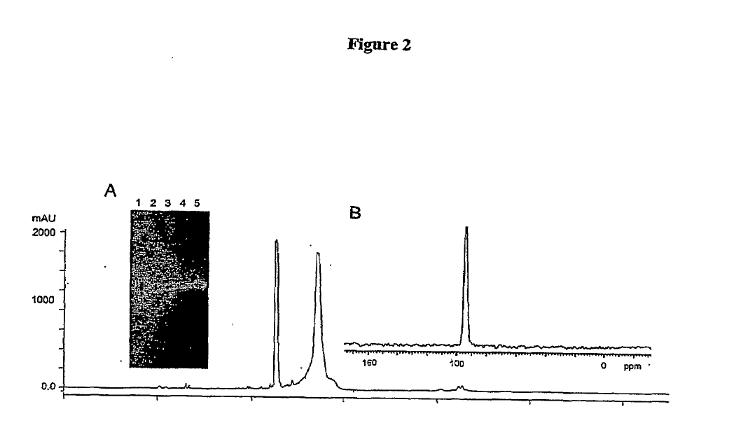

Purification was achieved by reverse phase HPLC. A typical result for a 10 mer (Compound 10, Table 2) having all four bases and borane phosphonate internucleotide linkages is shown in FIG. 2 (total reaction mixture). The major peak (excluding the first, anisic acid peak) is the product. As expected from many P-chiral centers and the resulting large number of stereoisomers, this peak is quite broad.

Numerous oligomers have been prepared using synthesis and purification strategy disclosed herein. Table 2 lists mass data for several examples having various combinations of 2′-deoxynucleoside bases and internucleotide phosphate and borane phosphonate linkages. As can be seen from the data, the observed masses for all ODNs correspond to those as calculated. Similarly, phosphorus NMR analyses display a broad signal at 96 ppm (borane phosphonate) and a sharp peak at −2 ppm when phosphate is part of the backbone. By phosphorus NMR, when all internucleotide linkages are borane phosphonate (Table 2, compound 10) phosphate cannot be detected (inset B, FIG. 2). ¹¹B NMR spectra for all oligomers consists of a broad signal at 40 ppm (relative to BF₃) which is characteristic of the borane phosphonate linkage. Neither the mass spectral nor boron NMR data suggest the presence of boronated bases or sugars. When total, unpurified reaction mixtures are analyzed by gel electrophoresis, only one major band which corresponds to the product is observed (inset A, FIG. 2). As expected from previous research, borane phosphonate ODNs are resistant to degradation by exonucleases (snake venom and calf-spleen phosphodiesterases) and DNase I.

TABLE 2

molecular weight

| No. | ODN^{a} | calculated | observed |
|---|---|---|---|
| 3 | d(TₚTₚT_{b})₄TₚT) | 4185.7 | 4183.4^{b} |
| 4 | d(T_{b}Tₚ)₆T_{b}T) | 4179.8 | 4177.5^{b} |
| 5 | d(GₚTₚG_{b}TₚGₚT_{b})₂GₚT) | 4360.8 | 4360.7^{b} |
| 6 | d(G_{b}TₚG_{b}T_{P})₃G_{b}T) | 4354.9 | 4355.2^{b} |
| 7 | d(T_{b})₉T | 2960.8 | 2954.5^{c} |
| 8 | d(A_{b}T_{b})₄A_{b}T) | 3005.9 | 3000.7^{c} |
| 9 | d(C_{b}T_{b})₄C_{b}T) | 2885.8 | 2881.5^{c} |
| 10 | d(T_{b}C_{b}T_{b}T_{b}A_{b}C_{b}T_{b}G_{b}A_{b}T) | 2973.9 | 2967.5^{c} |

^{a}p = phosphate; b = borane phosphonate;
^{b}Perseptive Biosystems Voyager Biospectrometry Workstation using a previously published procedure7;
^{c}HPLC-ESI-Q-TOF-MS Instrument System.

1. A compound having the formula: 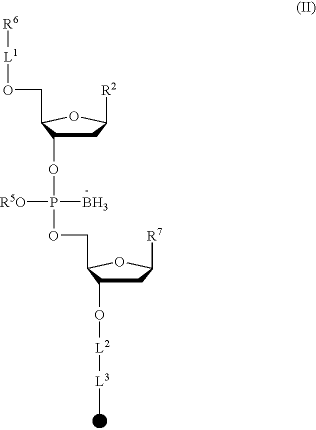
wherein,
R⁶ is hydrogen, or a silyl protecting group;
R² and R⁷ are independently selected from an N3 protected or unprotected thymine, an N2 protected or unprotected guanine, an N6 trityl protected adenine, or an N4 trityl protected cytosine, wherein at least one of R² and R⁴ are an N6 trityl protected adenine or an N4 trityl protected cytosine;
R⁵ is a fluoride ion compatible phosphorous center protecting group;
L¹ and L² are independently a bond or a 2′deoxy nucleic acid linker;
L³ is a base-labile solid support linker; and
the solid circle represents a solid support.

2. The compound of claim 1, wherein said N3 protected thymine is an N3 carboxyaryl protected thymine.

3. The compound of claim 1, wherein said N3 protected thymine is an N3 anisoyl protected thymine or an N3 benzoyl protected thymine.

4. The compound of claim 1, wherein said N2 protected guanine is an N2 carbamate protected guanine or an N2 trityl protected guanine.

5. The compound of claim 1, wherein said N6 trityl protected adenine is an N6 dimethoxytrityl protected adenine.

6. The compound of claim 1, wherein said N3 protected thymine is N3 anisoyl protected thymine

7. The compound of claim 1, wherein said N4 trityl protected cytosine is an N4 trimethoxytrityl protected cytosine.

8. The compound of claim 1, wherein said silyl protecting group is benzhydroxy-bis(trimethylsiloxy)silyl, bis(trimethylsiloxy)cyclododecyloxysilyl, or tris-(trimethylsiloxy).

9. The compound of claim 1, wherein R⁵ is methyl, benzyl, or cyanoethyl.

10. The compound of claim 1, wherein said 2′ deoxy nucleic acid linker is a polynucleotide or a single nucleotide.

11. The compound of claim 10, wherein said polynucleotide is an oligonucleotide.

12. The compound of claim 10, wherein said polynucleotide comprises a plurality of internucleotide linkages independently selected from a PIV posphonium borane adduct internucleotide linkage and a phosphate triester internucleotide linkage.

13. An N-trityl phosphoramidite molecule having the formula: 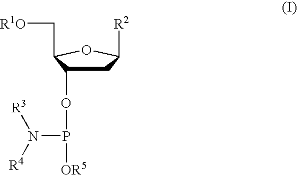
wherein
R¹ a silyl protecting group;
R² is an N6 trityl protected adenine, an N2 trityl protected guanine, or an N4 trityl protected cytosine;
R³ and R⁴ are independently selected from unsubstituted C₁-C₁₀ alkyl, substituted or unsubstituted aryl, or substituted or unsubstituted heterocycloalkyl; and
R⁵ is a fluoride ion compatible phosphorous center protecting group.

14. The compound of claim 13, wherein R³ and R⁴ are diisopropyl or morpholino.

15. A borane phosphonate nucleic acid compound comprising at least 20 nucleotides,
wherein at least one nucleotide is thymine, at least one nucleotide is guanine, at least one nucleotide is adenine, and at least one nucleotide is cytosine, and
wherein at least 50% of the internucleotide linkages are borane phosphonate linkages.

16. The borane phosphonate nucleic acid of claim 15, wherein every other internucleotide linkage is a borane phosphonate linkage.

17. The borane phosphonate nucleic acid of claim 15 comprising from 20 to 100 nucleotides.

18. The borane phosphonate nucleic acid of claim 15 comprising from 20 to 80 nucleotides.

19. The borane phosphonate nucleic acid of claim 15 comprising from 20 to 60 nucleotides.

20. The borane phosphonate nucleic acid of claim 15, wherein said nucleic acid is a 2′deoxy nucleic acid.

21. A pharmaceutical composition comprising the borane phosphonate nucleic acid of claim 15 and a pharmaceutically acceptable excipient.

22. A method of hybridizing the borane phosphonate nucleic acid of claim 15 to a complementary nucleic acid, said method comprising the step of contacting said complementary nucleic acid sequence with said borane phosphonate nucleic acid, wherein said complementary nucleic acid comprises a nucleic acid sequence having at least 50% base complementation relative to the borane phosphonate nucleic acid sequence.

23. The method of claim 22, wherein said complementary nucleic acid comprises a nucleic acid sequence having at least 80% base complementation relative to the borane phosphonate nucleic acid sequence.

24. The method of claim 22, wherein said complementary nucleic acid comprises a nucleic acid sequence having at least 90% base complementation relative to the borane phosphonate nucleic acid sequence.

25. The method of claim 22, wherein said contacting occurs in a mammal, said method further comprising, before said contacting, administering said borane phosphonate nucleic acid sequence to said mammal.

26. The method of claim 25, wherein said mammal is a human.

27. A method of synthesizing a borane phosphonate DNA, said method comprising the steps of:
a. contacting the 5′ hydroxyl of a solid phase 2′deoxy nucleic acid with the N-trityl phosphoramidite molecule of claim 13 thereby forming an N-trityl solid phase 2′ deoxy nucleic acid;
b. contacting said N-trityl solid phase 2′ deoxy nucleic acid with a boronation reagent thereby forming an N-trityl phosphonium borane solid phase 2′ deoxy nucleic acid;
c. contacting said N-trityl phosphonium borane solid phase 2′ deoxy nucleic acid with a fluoride ion thereby removing said silyl protecting group and forming a 5′-OH N-trityl phosphonium borane solid phase 2′ deoxy nucleic acid;
d. optionally extending said 5′-OH N-trityl phosphonium borane solid phase 2′ deoxy nucleic acid using one or more phosphoramidite coupling, 5′ deprotection, and oxidation cycles;
e. contacting said 5′-OH N-trityl phosphonium borane solid phase 2′ deoxy nucleic acid with an acidic reagent thereby removing the trityl protecting group and forming a 5′-OH phosphonium borane solid phase 2′ deoxy nucleic acid;
f. contacting said 5′-OH phosphonium borane solid phase 2′ deoxy nucleic acid with a phosphorous center deprotecting reagent to form a 5′-OH borane phosphonate solid phase 2′ deoxy nucleic; and
g. contacting said 5′-OH borane phosphonate solid phase 2′ deoxy nucleic with a base reagent thereby forming said borane phosphonate DNA.

28. A borane phosphonate nucleic acid compound comprising at least 10 nucleotides, wherein at least one nucleotide is thymine, at least one nucleotide is guanine, at least one nucleotide is adenine, and at least one nucleotide is cytosine, and at least one of the internucleotide linkages is a borane phosphonate linkage.
